(19) United States
(12) Patent Application Publication
(10) Pub. No.: US 2009/0068667 A1
(43) Pub. Date: Mar. 12, 2009

(54) METHODS AND ASSAYS FOR SCREENING STEM CELLS

(75) Inventors: Lorraine F. Meisner, Madison, WI (US); Julie A. Johnson, Madison, WI (US)

Correspondence Address: Casimir Jones, S.C., 440 Science Drive, Suite 203, Madison, WI, 53711, US

(73) Assignee: Cell Line Genetics, LLC, Madison, WI (US)

(21) Appl. No.: 12/202,913

(22) Filed: Sep. 2, 2008

Related U.S. Application Data
(60) Provisional application No. 60/969,482, filed on Aug. 31, 2007.

(51) Int. Cl. C12Q 1/68

(52) U.S. Cl. 435/6

(57) ABSTRACT

The present invention provides methods and assays for screening cells, such as stem cells, for chromosomal aberrations. In particular, the present invention provides a rapid, sensitive assay platform for detecting high and low levels of chromosomal aberrations present in a cell population. This includes, but is not limited to, detection of extra chromosomes (trisomies) as well as insertions of small segments that are undetectable using standard cytogenetic studies, wherein the abnormal cells comprise a low percentage of the total cell population.

The present application claims priority to U.S. Provisional Patent Application Ser. No. 60/969,482, filed Aug. 31, 2007, the entire disclosure of which is herein incorporated by reference in its entirety.

### FIELD OF THE INVENTION

The present invention provides methods and assays for screening cells, such as stem cells, for chromosomal aberrations. In particular, the present invention provides a rapid, sensitive assay platform for detecting high and low levels of chromosomal aberrations present in a cell population. This includes, but is not limited to, detection of extra chromosomes (trisomies) as well as insertions of small segments that are undetectable using standard cytogenetic studies, wherein the abnormal cells comprise a low percentage of the total cell population.

### BACKGROUND OF THE INVENTION

There is a recognized need in the industry for a rapid method for detecting significant genetic changes in both embryonic and adult stem cells, since human embryonic stem cell (hESC) lines have been shown to accumulate chromosome aberrations over time in culture (Draper et al., 2004, Nat. Biotech. 22:53-54; Maitra et al., 2005, Nat.Gen. 37:1099-1103). Molecular tests, such as gene expression arrays, SNPs (single nucleotide polymorphism genomic analysis), and mitochondrial DNA sequencing (Josephson et al., 2006, BMC Biol. 4:28; Nielsen et al., 2007, Regen. Med. 2:179-192, Maitra et al., 2005) are proposed technologies for characterizing stem cells. However, these technologies cannot identify abnormal cells that constitute less than 20% of the populations, whereas cytogenetics can detect aberrations in 5% of the cells and fluorescent in situ hybridization (FISH, for example see FIG. 5) can detect abnormal cells that comprise 0.5% or less of a cell line using interphase cells. However, commercially available FISH probes are not targeted to specific chromosomal regions that are likely to be trisomic in stem cells due to insertions or duplications resulting in partial trisomies.

Molecular biologists consider chromosome analysis to be a low read-out version of the genome that does not detect submicroscopic alterations in DNA (Maitra et al., 2005), but population wide gene changes are rare in cultured stem cells whereas extra copies of chromosomes 12 and/or 17, which are frequent findings in cancer, have been found in a large number of hESC lines. The importance of such chromosome changes have been well demonstrated in cancer, in which the driving force is the inherent instability of aneuploid karyotypes (Duesberg and Li, 2004, Cell Cycle 2:202-210). Unlike single gene mutations, chromosome aberrations involve blocks of genes that cause imbalance of many proteins and enzymes that can result in growth advantage, thereby providing a basis for selection and amplification of the abnormal cells. This enables these cells to proliferate more rapidly, replace the less aggressive normal cells and eventually dominate the culture. Once a chromosome change such as acquisition of an extra chromosome 12 or 17 occurs in a cell, it enjoys a selective advantage and can replace the normal cells in about 10 passages. It is important to detect such changes when only a few abnormal cells are present among a population of cells, so as to avoid use of contaminated cell populations in research, diagnostics, drug screening, and therapeutic applications.

As such, what are needed are sensitive methods and assays for screening for chromosomal aberrations in a stem cell population that will enable detection of abnormal cells present in less than, for example, 0.5% of the cell population.

### SUMMARY OF THE INVENTION

The present invention provides methods and assays for screening cells, such as stem cells, for chromosomal aberrations. In particular, the present invention provides a rapid, sensitive assay platform for detecting high and low levels of chromosomal aberrations present in a cell population. This includes, but is not limited to, detection of extra chromosomes (trisomies) as well as insertions of small chromosome segments (partial trisomies) that are undetectable using standard cytogenetic studies, wherein the abnormal cells comprise, for example, from 0.5% or less of the population.

Certain illustrative embodiments of the invention are described below. The present invention is not limited to these embodiments.

Methods and assays for rapid screening of stem cell lines for the presence of specific chromosome aberrations typical of stem cells (e.g., embryonic, adult, reprogrammed (e.g., induced pluripotent stem cells), etc.) of different species used in stem cell research and therapy is described herein. In some embodiments, assays comprise slide arrays or other surface or bead-based arrays with DNA probes and normal control cells for each stem cell species to be tested. In some embodiments, the present invention provides slide arrays with appropriate probes for use in stem cell cultures, with such probes containing those DNA sequences that are specifically amplified or deleted in different types of cancer (e.g., breast, colon, pancreas, lung etc.). Assays of the present invention provide for detection of chromosomal aberrations in low levels of cells (e.g., 0.5% or less) with the targeted abnormal chromosomes.

In some embodiments, stem cells that are grown in vitro, passaged, and aliquoted into multiple tubes, are tested using systems and methods of the present invention. An embryonic stem cell culture in which one cell out of 200 is found to have an extra copy of chromosome 12 or 17 may have an extra copy in essentially 100% of the cells even in ten passages or later (and may represent 50% of the cells after as few as four passages). As such, criteria developed for interpreting significant changes in clinical cytogenetics, in which a single abnormal cell in 20 is regarded as an artifact, cannot be applied to stem cell cultures, wherein finding an extra copy of specific chromosomes, or part of those chromosomes, can be highly significant. When low level aberrations have been identified in a stem cell line, researchers can go back to an earlier passage of that line to find a completely normal population, and in these cases, 500 interphase nuclei can be rapidly screened to ensure that the cell line is normal since the aberration was not present in, for example, 0.2% of the cells. Due to limited availability of trained cytogeneticists, who are generally employed by clinical laboratories, cytogenetics analysis is costly and, on average, takes at least two months due to competition with clinical studies. In contrast, the assays and methods as described herein, unlike standard cytogenetics, are performed by individuals with minimal training, or can be adapted for automated formats thereby saving time and expense. The methods and systems of the present invention are applicable to stem cells from any desired species, including but not limited to humans (e.g., non-human primates, non-human mammals, etc.). For example, much stem research is conducted using rodent (e.g., mouse, rat, etc.) stem cells.

In some embodiments, the present invention provides for specific locus probes designed for use with interphase stem cells, to detect chromosomal aberrations. Probes of the present invention are created, for example, from BAC cloning, yeast artificial chromosome (YAC) cloning, microdissection of metaphase chromosomes, PCR amplified DNA sequences, synthetic oligonucleotides and synthesized peptide nucleic acids (PNAs), etc. In some embodiments, probes used in assays of the present invention are directly or indirectly labeled with a fluorescent moiety for single fluorescence measurement. As well, multiple probes, each labeled with different fluorescent moieties of different wavelengths, may be used such that fluorescent signal multiplexing can occur in one area, targeting one or more chromosomes, thereby maximizing a stem cell sample being assayed and space on the assay substrate. In some embodiments, probes used are indirectly labeled with non-fluorescent moieties, such as digoxigenin, peroxidase or biotin, which can be conjugated to an antibody, avidin, etc., for detection using light microscopy. In one embodiment, the probes are made from synthesized peptide nucleic acids and labeled with digoxigenin. Fluorescent moieties useful in systems and methods of the present invention include, but are not limited to, cascade blue, coumarin, cyanine dyes (e.g., 2, 3, 5, 7), BODIPY dyes (e.g., FL, TMR, TR, 650/665) Alexa dyes (e.g., 488, 532, 546, 594), fluorescein isothiocyanate (FITC), spectrum green, tetramethylrhodamine (TMR), rhodamine B, spectrum orange, texas red, and spectrum red. Commerically available FISH probes, such as painting probes, only identify anolomies on metaphase chromosomes and not interphase chromosomes. Some embodiments of the present invention are directed to a rapid method for interphase detection of chromosomal anomolies, which does not require, for example, tissue culture, etc. and other time steps necessary for maintaining actively dividing cells.

In some embodiments, an assay of the present invention comprises a substrate such as a slide (e.g., glass, polypropylene, polyethylene, etc,), a membrane surface, and the like. The assay substrate comprises multiple discrete locations for the application of different stem cell species or control cells on each discrete location. FIG. 2 depicts an exemplary substrate (slide) for use in an assay of the present invention. In some embodiments, samples from stem cell species for assay and control cells are taken from aliquots of passaged cells and placed each on a discrete location on an assay substrate. The labeled probes are subsequently added to each location on the substrate, and in situ hybridization is performed. Hybridization results are detected by any detection means, such as fluorescence (fluorescently labeled probe) microscopy and light microscopy (non-fluorescently labeled probe), either system being adaptable to an automatic reader.

In some embodiments, an assay of the present invention comprises two substrates, or slides. One slide comprises, for example, discrete locations with samples from aliquoted control cells and stem cell species to be tested, and the second slide comprises labeled probes capable of hybridizing to one or more chromosomal aberrations. In this embodiment, hybridization buffer is applied to the probe slide that is laid atop the cell-containing slide, followed by hybridization and detection methods known to those skilled in the art (FIG. 2A). In some embodiments controls are used for comparison. For example, if the specimen to be tested has clear signals, with minimal cell overlapping, reliability of the results is possible by comparing the signal counts to those of the normal control cells that undergo the same hybridization conditions. As long as the normal control demonstrates two signals for the probe being assayed, finding signals greater than two in any of the stem cells being tested is considered evidence for low level trisomy. Scoring may also be based on cut-off values that are developed after scoring at least ten cultures with a specific probe. Also, when both probes show the same frequency of cells with four signals, it is suggestive of the presence of a low level of cells with teteraploidy. If low or high level trisomy of either probe is detected, or tetrasomy 12 due to formation of an isochromosome (two copies of the short-arm with loss of the long-arm) leading to duplication of the critical region of 12 to which the probe is directed, this would prompt the investigator performing the assays to go to an earlier normal passaged aliquot. A potential for misinterpretation, for example, is in the case of low level tetraploidy of both probes on the slide array, in which case a few cells would show four signals for chromosome 12 while a similar number would show four signals for 17. When both probes show the same frequency of cells with four signals strongly suggesting the presence of a low level of cells with tetraploidy, a slide array using a control probe for a genetic sequence unlikely to undergo in vitro selection would be provided for either quantifying, or ruling out, tetraploidy.

In some embodiments, the present invention provides assay kits. Assay kits of the present invention comprise a substrate, such as a slide, and pre-measured aliquots of reagents needed for hybridization and post-hybridization reactions. Reagents needed for hybridization and post-hybridization reactions include, but are not limited to, directly or indirectly labeled probes with or without blocking DNA (e.g., human placental DNA, salmon sperm DNA, etc.) or similar reagents (e.g., pepsin, protease, etc.), hybridization wash solutions (e.g., SSC, etc.), post-hybridization solutions (e.g., SSC with detergents, etc.), counterstains (e.g., propidium iodide, DAPI, etc), and the like. An example of the use of blocking DNA in FISH for chromosomal staining can be found in U.S. Pat. No. 5,447,841 (incorporated herein by reference in its entirety). In some embodiments, the substrate of the kit comprises pre-deposited control cells, wherein an investigator adds the stem cell species for testing. In some embodiments, an assay kit further provides labeled probes for adding to the substrate upon which is located the cells for testing (e.g., test and control cells). In other embodiments, the substrate comprises pre-deposited labeled probes, wherein the investigator adds the stem cells for testing and control cells (which can be additionally furnished in the assay kit). In some embodiments, an assay kit of the present invention comprises two substrates, one of which comprises pre-deposited control cells that have already been added to the labeled probes, and the second of which comprises pre-deposited labeled probes to which the stem cells are to be added.

In some embodiments, additional buffers, solutions, specialized slides or substrates, detection reagents, and the like necessary, sufficient or useful to perform washes, hybridizations, counterstaining, etc. are furnished in an assay kit of the present invention. An assay kit further comprises instructions for performing the assay, as well as scoring criteria for determining chromosomal aberration presence or absence and amount thereof, if present. In some embodiments, software is provided that is configured to analyze, store, correlate, or otherwise manipulate data obtained from use of the assay. In some embodiments, the software associates information about the source of the stem cell with information pertaining to the presence, absence, nature of, or level of aberration. In some embodiments, data obtained is compared to data in a database so as to predict or characterize a test sample based on its similar properties to a stored database sample. In some embodiments, the kit comprises detection equipment. In some embodiments, the kit provides a desktop or handheld device that carries out one or more of the steps useful in utilizing the invention, including, but not limited to, sample preparation, sample processing/incubation, assay preparation, assay use, label detection, software, data collection, data storage/analysis, and the like.

The present invention further provides probes or sets of probes particularly useful in identifying chromosomal aberrations in stem cells, as well as compositions comprising such probes (e.g., kits, reaction mixtures, slides, beads, etc.). The probes may hybridize to and detect a critical region that identifies all of the translocations and duplications associated with the most common chromosomal aberrations found in stem cell lines. Aberrant cells identified by the probes may be discarded. Cells lacking aberrations may be used for any number of purposes, including, but not limited to, maintenance of a cell lines, research, drug screening, transplantation into an organism for research or therapy, and the like.

### DESCRIPTION OF THE FIGURES

FIG. 1 shows exemplary cytogenetic results of 155 human embryonic stem cell cultures processed within a nine-month period.

FIG. 2 shows an A) exemplary critical region array “sandwich” design for chromosome 12 and 17 critical regions, and B) an exemplary kit comprising a slide, control cells and areas for adding the stem cells to be tested. A further embodiment of the kit includes probes for the species being tested.

FIG. 3a shows an exemplary chromosome 17 probe map delineating a critical region for a probe or probes that identify all partial or full trisomies of chromosome 17. Probes used to identify critical region sequences are shown along with the results of probing as described in Example 3, below. FIG. 3b shows a corresponding map for chromosome 12.

FIG. 4 shows an embryonic stem cell karyotype with a cryptic duplication of the critical region of chromosome 17

FIG. 5 shows A) a standard FISH test for trisomy 17 in which using probes for centromere 17, HER-2/neu and Topo 2 did not identify the chromosome 17 material translocated to chromosome 21, and B) using a chromosome 17 FISH paint probe shows that the unknown extra material on chromosome 21 is derived from 17.

FIG. 6 shows the frequency of normal and abnormal karyotypes in approximately 230 murine cell lines, showing the frequency at which each probe combination can identify cell lines with trisomies of 8, 11, X, Y, 6, or 12.

FIG. 7 shows the frequency of normal and abnormal karyotypes in approximately 700 human cell lines, showing the frequency at which each probe combination can indentify cell lines with abnormal karyotypes. Cell lines positive for trisomies of 1q and/or 20 are those that were not positive for trisomy 12 and/or 17. Likewise, cell lines positive for trisomy 8 and/or 13 are those that were not positive for 12/17/1q/20.

### DEFINITIONS

As used herein, the term “hybridization” is used in reference to the pairing of complementary nucleic acids. Hybridization and the strength of hybridization (i.e., the strength of the association between the nucleic acids) is impacted by such factors as the degree to which the nucleic acids are complementary, stringency of the conditions involved, the Tₘ of the formed hybrid, and the G:C ratio within the nucleic acids.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

As used herein, the term “probe” refers to a specific sequence, whether occurring naturally as in a purified restriction digest or produced synthetically, recombinantly or by PCR amplification or nick translation, that is capable of hybridizing to at least a portion of another nucleic acid of interest. A probe may be single-stranded or double-stranded. A probe may be a mixture of two or more probes, each labeled with a different label moiety (e.g., different fluorophors) and each targeting a different region of the same chromosome, or a mixture targeting the critical regions of different chromosomes. Probes are useful in the detection, identification and isolation of particular gene sequences. Probes are typically labeled and a detection moiety and detection is available with any detection system, including, but not limited to enzyme (e.g., ELISA, as well as enzyme-based histochemical assays), fluorescent, radioactive, calorimetric, luminescent and other visible systems. It is not intended that the present invention be limited to any particular detection system or label.

A used herein, the term “stem cell” refers to primal cells found in all multi-cellular organisms that retain the ability to renew themselves through mitotic cell division and can differentiate into a diverse range of specialized cell types. Three broad categories of mammalian stem cells are: (1) embryonic stem cells derived from blastocysts, (2) adult stem cells which are found in fetal tissues, umbilical cord blood and in specific niches within adult tissues, and (3) reprogrammed adult cells engineered to de-differentiate to an embryonic stem cell-like pluripotent state. In a developing embryo, stem cells can differentiate into all of the specialized embryonic tissues. In adult organisms, stem cells and progenitor cells often act as a repair system for the body, replenishing specialized cells. The present invention is based on the evaluation of interphase nuclei, and not dividing cells, and is contemplated for use with all stem cells, regardless of origin, unless otherwise noted.

As used herein, the term “clone” refers to a subpopulation of cells with chromosome changes that distinguish it from the original cell line. A stem cell line may have two or more clones, one of which may be normal and the other(s) of which have chromosome aberrations that differentiate each clone from the original stem cell line.

As used herein, the term “solid surface” refers to any solid surface suitable for the attachment of biological molecules and the performance of molecular interaction assays. Surfaces may be made of any suitable material (e.g., including, but not limited to, silicon, plastic, glass, polymer, ceramic, photoresist, nitrocellulose, hydrogel, paper, polypropylene, polystyrene, nylon, polyacrylamide, optical fiber, natural fibers, nylon, metals, rubber and composites or polymers thereof) and may be modified with coatings (e.g., metals or polymers). Furthermore, a solid surface may comprise two or more materials (e.g., glass and nylon). Solid surfaces need not be flat. Solid surfaces may include any three dimensional shape including pins, rods, fibers, tapes, threads, sheets, films, gels, membranes, beads, plates, particles, microtiter wells, capillaries, or cylinders. Materials attached to solid surfaces may be attached to any portion of the solid surface (e.g., may be attached to an interior portion of a porous solid support material). Additionally, the solid surface (e.g., glass) may be treated (e.g., amine or epoxy treated) for use in the present invention. Preferred embodiments of the present invention have biological molecules such as nucleic acid molecules attached to solid surfaces. The term “attached,” when used to describe a state of interaction between a biological material and a solid surface, describe non-random interactions including, but not limited to, covalent bonding, ionic bonding, chemisorption, physisorption and combinations thereof.

As used herein, the term “sample” is used in its broadest sense. In one sense, it is meant to include a specimen or culture obtained from any source, as well as biological and environmental samples. Biological samples may be obtained from animals (including humans) and encompass fluids (e.g., blood or urine), solids, tissues, and gases. Such examples are not however to be construed as limiting the sample types applicable to the present invention.

The terms “test compound” and “candidate compound” refer to any chemical entity, pharmaceutical, drug, and the like that is a candidate for use to treat or prevent a disease, illness, sickness, or disorder of bodily function. Test compounds comprise both known and potential therapeutic compounds.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature.

### DETAILED DESCRIPTION OF THE INVENTION

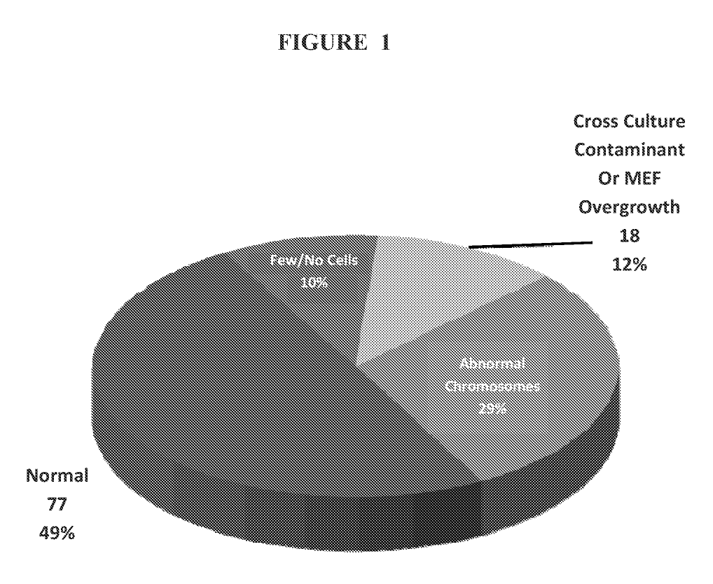

An important consideration in embryonic stem cell culture is the presence of aberrations in copy number of chromosomes commonly observed in cancer (e.g., such as extra copies of chromosomes 12 and/or 17 in human lines). Although these aberrations may be detectable with SNP arrays (Maitra et al, 2005), it is known that at least 20% of a cell population must carry a genetic abnormality before it can be detected on a SNP array (Josephson et al., 2006). On the other hand, cytogenetic analysis detects such changes if they are present in at least 5% of the culture, and FISH (fluorescence in situ hybridization) detects extra copies of chromosme 12 or 17 if present in at least 0.5% of the cell population (Baker et al., 2007, Nat. Biotech. 25:207-215). However, the ability to follow hESC with cytogenetics is limited by the scarcity of trained cytogeneticists available for such testing, whereas an array of the present invention enables testing to be performed in house by technicians with limited experience using instrumentation typically available in stem cell laboratories. FIG. 1 shows results from cytogenetic testing of 155 human embryonic stem cell cultures processed within a nine-month period. As can be seen, cell abnormalities are found in a large part of the cell cultures. Sensitive screening for these abnormalities is necessary to establish good from bad cultures.

The situation for detecting chromosome aberration in mouse or monkey embryonic stem cells is even more acute than for human ESC cultures, since cytogeneticists in clinical laboratories are generally unable or unwilling to analyze monkey or mouse chromosomes. To date, most embryonic stem cell research has involved mouse cells, further emphasizing the importance of detecting chromosome aberrations in those cells, especially since mouse chromosomes are even more likely to develop chromosome abnormalities than human ESC (see Table 1).

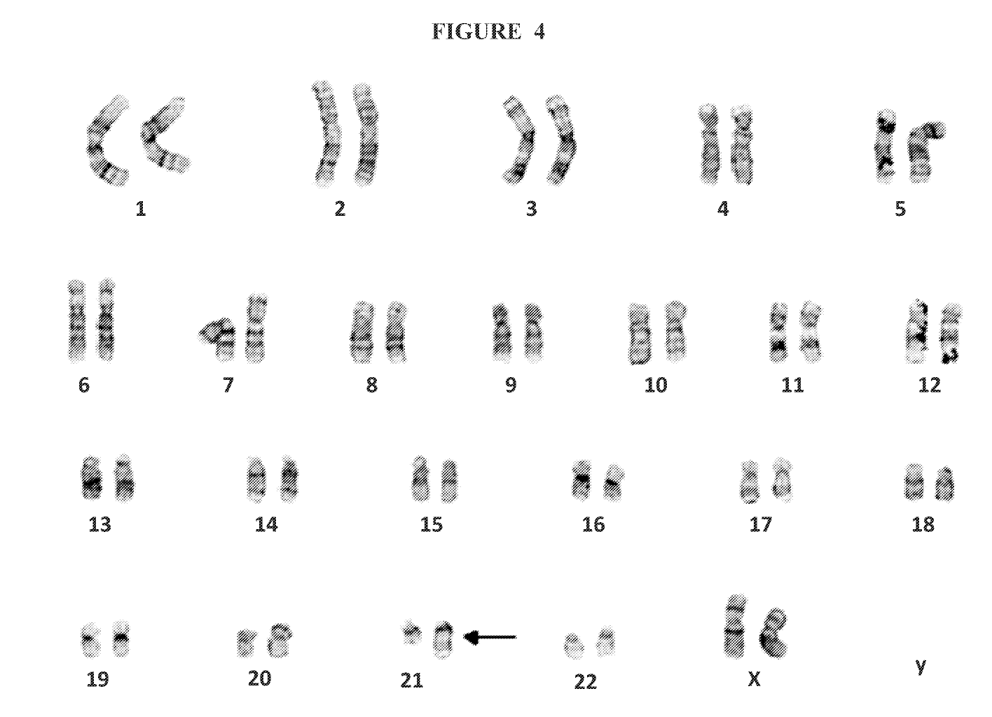

Embodiments of the present invention provide a simple method for testing hESC lines for significant trisomies or partial trisomies before they are used for, for example, research or therapeutic purposes. Embryonic stem cells and cancer cells have much in common, including indefinite self-renewal, loss of contact inhibition, anchorage independence, and an increased capacity for proliferation. Therefore it is not surprising that the most frequent aneuploidies found in hESC are trisomies of, for example, chromosome 12 and/or 17. Human germ cell tumors are characterized by extra copies of all or part of chromosome 12, and extra copies of all or part of chromosome 17 (FIG. 4) appear to be associated with a proliferative advantage in many cancers. This emphasizes the need to screen for such changes in hESC before the abnormal cell lines are expanded for research or therapeutic applications. Also, many of the proteins expressed by embryonic stem cells (e.g., OCT4, SSEA, NANOG) are identical to those in cancer, and hESC cultures with trisomy 12 or 17 have typical embryonic protein expression despite having a proliferative advantage (Mitalipova et al., 2005, Nat. Biotech. 23:19-20). In some embodiments, screening involves other chromosomes in hESCs, such as aberrations of chromosomes 1 (e.g., band 32), 12, 13, 18, 20 and/or X, or specific regions within these chromosomes (for example, bands q21-32 of chromosome 1 or 18q21) that are determined to be aberrant in the course of studying a large number of human ESC cultures from different sources. For example, as medium and cell culture techniques evolve, other chromosome changes are contemplated to be important and these will be added to the screening assay(s). For example, X chromosomal gain is a consistent finding in testicular germ cell tumors. After examining gene expression patterns on the X chromosome, Kawakami et al. (2003, J. Urology 169:1546-1552) observed that multiple X chromosomes in testicular germ cell tumors were predominantly hypomethylated and active regardless of expression of XIST, the gene associated with inactivation of all X chromosomes except for one of them. A biological significance of excess active X chromosomes in testicular germ cell tumors is, for example, enhanced expression of two X-linked oncogenes ARAFI and EKK1 in the germ cell tumor cell lines.

Flexibility in determining different chromosomal anomalies is further provided by embodiments of the present invention. Such flexibility is important, for example, as new anomalies become apparent and important. It is contemplated that, in some instances, chromosomal anomalies arise due to differences in the culture conditions hESCs are exposed to. For example, it is reported by Allegrucci & Young, 2007, Hum. Repro. Update 13:103-120 that many differences that are emerging between hESC lines may be more associated with the wide range of culture conditions used than inherent genetic variation of the embryos from which the lines were derived. As such, flexibility is important as an embodiment of the present invention to provide for accurate determination of chromosomal anomalies due not only to inherent genetic variation, but also anomalies arising from culture conditions or experimental manipulation of cells used in therapeutic stem cell applications.

Characteristic chromosome changes have also been found in embryonic stem cells from other species. For example, mouse cell lines tend to acquire extra copies of chromosomes 8 and 11, while rhesus macaque lines demonstrate trisomy of 11 and 16 (which are in large part homologous to human 12 and 17). About 25% of all cell lines studied in developing embodiments of the present invention had two or more clones, with minor clones often constituting less than 10% of the population. As such, the present invention as described herein is designed to provide a rapid means of detecting low level populations with the chromosome changes characteristic of different species by providing species specific screening tools. While it is likely that many different chromosome aberrations occur in vitro as well as in vivo, trisomies of (including, but not limited to) 12 and 17 provide a selective proliferative advantage whereas cells with non-adaptive changes are rapidly eliminated in vitro. Other adaptive aberrations will also be screened for (e.g., trisomy 20, 18 deletion, duplication of bands 1q21-24, etc.).

In developing embodiments of the present invention, based on initial data in studying over 500 embryonic stem cell lines, it was observed that almost 50% of human cultures yielded normal cytogenetics results (see FIG. 1), while in mouse ESC cultures only 22% had normal karyotypes (Table 1).

TABLE 1
Clonal cytogenetic findings in 100 mouse embryonic stem cell lines

| Normal | Pseudo- | Trisomy | Trisomy | Y | | Trisomy | Other | Deleted | Other |
|---|---|---|---|---|---|---|---|---|---|
| Karyotype | diploid² | 8 or i(8)³ | 11, i(11) | loss | i(Y)² | 6 or 9 | Trisomy | 10 | Deletions |
| 22 | 18 | 37 | 20 | 15 | 3 | 7 | 3 | 5 | 7 |

Pseudodiploid (²) cell lines have a normal chromosome count but an abnormal karyotype due to extra copies of one chromosome but missing another, or intrachromosomal rearrangements.
Isochromosomes (³) consist of two copies of a chromosome fused at the centromere.

Certain illustrative embodiments of the invention are described below. The present invention is not limited to these embodiments.

It is contemplated that stem cell lines develop species-specific chromosome aberrations like those found in cancer in vivo, and it is the intention of embodiments of the present invention to provide a rapid means of screening for such aberrations. The proposed methods and assays provide a sensitive platform for detecting low level clones, which is not possible with gene arrays or other technologies.

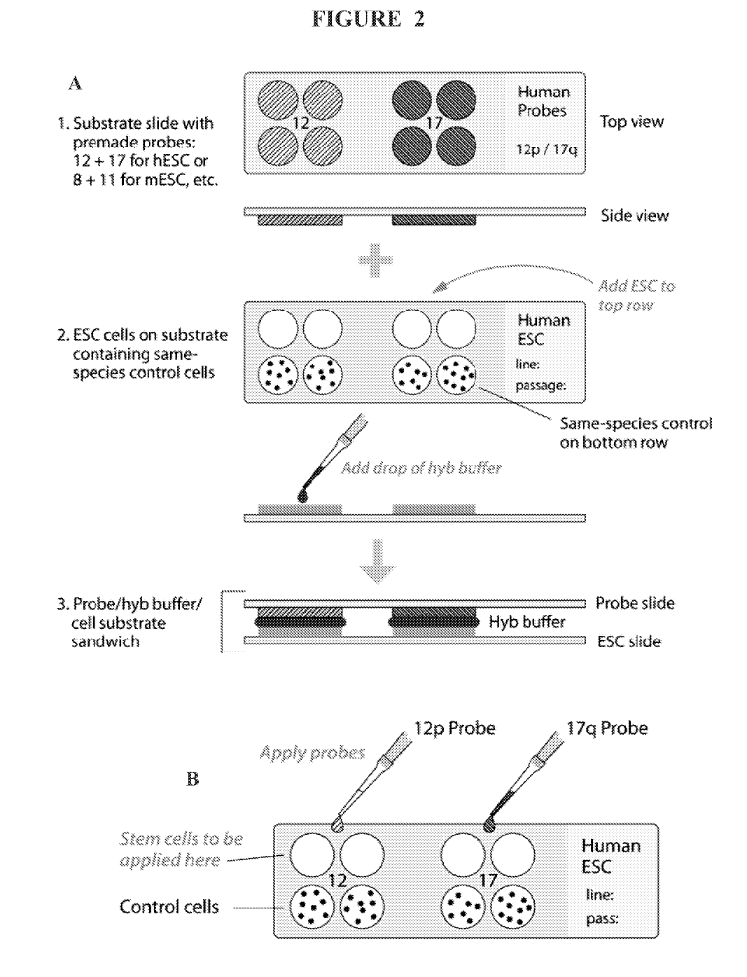

In one embodiment, an assay of the present invention comprises a glass slide or other solid surface with a plurality (e.g., eight) of zones, half on the top and half on the bottom of the surface (e.g., slide) (FIG. 2). In each zone resides one or more probes specific for human embryonic stem cell anomalies. For example, a probe for the specific region of chromosome 12 related to germ cell tumors (e.g., the “critical region”) is placed in the four left circles as shown in FIG. 2, whereas a probe for the critical region of chromosome 17 is placed in the four right circles. The probe is constructed, for example, by microdissection to target regions on metaphase chromosomes in order to obtain chromosomal material from the critical region, followed by PCR and labeling. An additional technique for probe construction is to use bacterial artificial chromosome (BAC) clones containing DNA sequences falling within bands 12p11.2 and/or band 12p13.3. Examples of BAC clones for probes includes, but is not limited to, D12S2005, D12S1640, D12S333, and/or D12S336 (all from 12p11.2) and/or D12S314, D12S93, D12S32S and/or D12S1690 (all clones from 12p13.3). Probes from these regions identify trisomy 12, isochromosome 12p, and partial duplications of the 12p critical region. If an extra copy of the critical region of chromosome 12 is present in a cryptic translocation, it is also detected with the probe, including the region containing the two most proximal genes on the 12 short-arm if adequate visualization of hybridization is possible.

For the bottom half of the surface, a probe cocktail specific for the critical region on the long-arm of chromosome 17 is placed on the four circles on the bottom half of the slide. The probe is constructed, for example, from BAC clones falling within the chromosome 17q23.1-q25.3 critical region containing genes that are amplified in both human embryonic stem cells and human cancers. Examples of BAC clones for probes include, but are not limited to, D17S794, D17S807, IB754, and/or WI-7837. The exact make-up of the final probe is based on those sequences yielding the highest sensitivity and specificity when tested on appropriate positive and negative stem cell lines.

The present invention provides methods and systems comprising probes targeting the genetic sequence yielding the highest sensitivity for, for example, determining trisomy and/or other chromosomal aberrations. This is performed for other chromosome aberrations that occur to a lesser frequency in human cells including, but not limited to those involving chromosome 1, 12, 13, 18, 20 and/or X. It is contemplated that future research will identify other chromosome aberrations, and these are included as targets in methods and systems of the present invention. The same or similar procedures are applied to mouse and monkey embryonic stem cells, targeting chromosomes 8 and 11 in the mouse, and 11 and 16 in the monkey, as well as other significant chromosome aberrations (e.g., segments in mouse embryonic stem cells from chromosomes 6 and/or 9). However, the chromosome regions to be targeted are contemplated to change with new types of media or protocol changes, and as these are consistently detected the assays can be modified for their inclusion.

In some embodiments, known normal cells are seeded along with unknown stem cells on a slide (FIG. 2) and serve as a normal control. In another embodiment, two slides are used, with probes placed on one slide and stem cells to be tested on another, wherein the two slides are subsequently sandwiched together. In some embodiments, hybridization buffer and/or other reagents are added to one of the slides, for example, prior to sandwiching the two slides together. Normal cells are further provided on the slide containing the stem cells to serve as a control for hybridization efficiency and counting accuracy. In some embodiments, the stem cell samples to be tested are taken from aliquots of passaged stem cells, whereas in other embodiments the stem cell samples to be tested are taken directly from cell culture dishes (e.g., actively growing stem cell cultures either from passage number 2 or 3, or stem cells that had been previously passaged and aliquoted for storage, but now are being actively grown and expanded).

In some embodiments, the sandwich assay design as described above is furnished in a kit. In such an embodiment, a sandwich style kit comprises two slides; one comprising labeled probes and one for addition of the normal control and the unknown stem cells by the user. Further included in the kit are, for example, hybridization buffers, blocking DNA or similar reagents (e.g., pepsin, protease, etc.), hybridization wash solutions (e.g., SSC, etc.), post-hybridization solutions (e.g., SSC with detergents, etc.), counterstains (e.g., propidium iodide, DAPI, etc), and the like and instructions for scoring the hybridization reactions and determining compromised stem cell populations from uncompromised populations. In some embodiments, a kit for assaying for chromosomal anomalies in stem cells comprises one slide such as that found in FIG. 2B, for example. Such a kit further comprises, for example, aliquots of labeled probes, hybridization buffers, blocking DNA or similar reagents (e.g., pepsin, protease, etc.), hybridization wash solutions (e.g., SSC, etc.), post-hybridization solutions (e.g., SSC with detergents, etc.), counterstains (e.g., propidium iodide, DAPI, etc), and the like and instructions for scoring the hybridization reactions and determining compromised stem cell populations from uncompromised populations.

In some embodiments, methods and assays of the present invention provide for the diagnosing of compromised stem cell populations. In some embodiments, diagnosing a compromised stem cell population comprises scoring for fluorescent hybridization signals that is performed following hybridization of the labeled probes to the stem cell populations being tested, for example as described above. Said scoring is used to determine a true positive stem cell population (e.g., one with a chromosomal anomaly and thus a compromised stem cell population). The methods of embodiments of the present invention are highly sensitive in making such a determination.

In some embodiments, a scoring system is used whereby a predefined number of signals (corresponding to aberrant cells) detected among a population of cells defines the population as problematic, potentially problematic, or non-problematic. The particular number of signals used to define the threshold may vary from cell type to cell type and application to application. In some embodiments signal loss (only one signal) or gain (more than two signals) represents a problematic cell population. In some embodiments less than 0.5% (e.g., 1 out of 200 cells) defines non-problematic cultures or cell lines and greater than 1% (e.g., 2 out of 200 cells) defines potentially problematic or problematic cultures. In some embodiments, 1.5% of a cell population (e.g., 3 out of 200 cells) or more is defined as problematic, 1% or more is potentially problematic, and less than 1% is non-problematic. In some embodiments, higher numbers of signals are used to set the thresholds (e.g., 4 out of 200 cells, 5 out of 200 cells, 6 out of 200 cells, . . . ). Defining these different characteristics allows a practitioner to select the fate of the cells. For example, cell populations defined as non-problematic are used (e.g., for research, therapeutic or other uses) and problematic cell populations are discarded. In some embodiments, potentially problematic cell populations may be used (for example, for drug testing or to simulate cancer evolution) or discarded. However, in some embodiments, the cells are further passaged (e.g., 5 passages, 10 passages, etc.) and retested. If the retested cells are problematic, the culture or cell line is discarded.

Experiments conducted during the development of embodiments of the present invention identified the following exemplary process as useful for screening stem cells. If a stem cell population has cells with three or more signals (e.g., 3 cells in 200 (3/200), 3/400, 3/500, etc.), for example using probes specific to the critical region of chromosome 17 or other aberrations described herein or later identified, then that population is deemed a potential true positive and further use is ceased. A potential true positive stem cell population may be further passaged further for a defined number of passages, for example ten passages, and retested. If cells with an extra signal have increased above two (e.g., 3/200, 4/300, 5/200, 5/300, 5/400, etc.), then the stem cell population is deemed a true positive and use of this particular population and any of its frozen or stored aliquots is stopped. If a stem cell population being tested shows no increase in fluorescent signals following hybridization, then that stem cell population is deemed negative for the tested chromosomal anomaly and is cleared for further use (see Example 1 for exemplary testing performed for subsequent scoring).

In some embodiments, after hybridization (which may use microwave to accelerate the reaction) the probes are detected by commonly used procedures, such as fluorescence (e.g., fluorescent in situ hybridization or FISH). If several probes comprise different fluorescent labels (e.g., with different wavelength maxima), then multiplexing is possible and, for example, probes for the 12 and 17 critical regions can be located in one area, thereby increasing assay efficiency and use of substrate space. Muller et al., CSH Protocols; 2007; doi: 10.1101/pdb.prot4730 describes protocols for the preparation of multiple fluorescent FISH probes.

In preferred embodiments, non-fluorescent hybridization is utilized by labeling the probes with digoxigenin, biotin or myeloperoxidase or similar labels thereby enabling detection with a standard light microscope (Chevalier et al., 1997, J. Histo. Cyto. 45:481-491). The use of a light microscope further provides the investigator a format for counting the signals in interphase nuclei of the test specimen, as well as the control specimen, using a single color.

In some embodiments, labels (e.g., paper, bar codes, etc.) are further affixed to the back of the slides for identification purposes of the stem cells, control cells, and probes used. In some embodiments, a slide is provided an investigator wherein said slide already comprises control cells in one or more of the regions or circled areas (wells). As such, the investigator only need add the stem cells for testing. As well, in some embodiments a slide is provided an investigator wherein said slide already comprises the probes affixed to the slides. In some embodiments, using non-fluorescent probes or FISH with a single probe color, the signals will look the same unless different fluorophores (e.g., with different wavelengths of detection) are affixed to the different probes, such as different colors for the probes for 12 or 17. In such a case, the probe numbers are affixed to the slide to enable the investigator to know which chromosome they are counting. This simple lay-out is adaptable for automated counting.

In some embodiments, the present invention provides a means for detecting probe and target hybridizations. When using a direct labeled fluorophore or a hapten (e.g., biotin, digoxigenin) requiring fluorescent detection, a fluorometer is used. Fluorometers include, but are not limited to, fluorescent plate readers, fluorescent microscopes, etc. When the probe is indirectly labeled with a hapten and detected by conjugation with enzymes that result in a visible substrate precipitation, a light microscope, for example, provides a simple means for detection. A skilled artisan will recognize all the options for probe labeling and detection systems that would be useful in detecting probe/target hybridizations.

In some embodiments, bacterial artificial chromosomes (BACs) that carry the genetic sequences of interest are used as cloning vectors for constructing the probes. The DNA from the BACs is isolated and purified using standard techniques, followed by labeling with fluorescent or non-fluorescent dyes (e.g., biotin, digoxigenin). After validating the analytical sensitivity and specificity of the probes using normal and abnormal stem cells, the best probes are selected.

In some embodiments, peptide nucleic acid synthesis is used for constructing probes for use with assays of the present invention. Peptide nucleic acids (PNA) are DNA mimics with a pseudopeptide backbone. PNA is an extremely good structural mimic of DNA (or RNA), and PNA oligomers are able to form very stable duplex structures with Watson-Crick complementary DNA, RNA (or PNA) oligomers, and they can also bind to targets in duplex DNA by helix invasion. As such, PNA probes can be used on denatured or non-denatured targets. Protocols for creating PNA probes are found in, for example, Nielsen et al., 2004, Peptide Nucleic Acids: Protocols and Applications, Horizon Bioscience Publishers, incorporated by reference herein in its entirety. In some embodiments, PNAs are labeled with fluorescent moieties, or preferably non-fluorescent moieties (e.g., digoxigenin).

Procedures for the making and detecting of probes for in situ hybridization are found in, for example, Schwarzacher and Helsop-Harrison, 2000, Practical in situ Hybridization, Springer-Verlag, BIOS Scientific Publishers, incorporated by reference herein in its entirety. Schwarcher 2000 discusses using fluorescent and non-fluorescent probes (biotin or digoxigenin) for assessing the incorporation of label on membrane. Protocols for making indirectly labeled non-fluorescent probes with, for example, biotin or digoxigenin, are found in Langer et al., 1981, Proc. Natl. Acad. Sci. 78:6633-6637, incorporated by reference herein in its entirety.

In some embodiments, assays of the present invention comprise slides for screening rhesus macaque embryonic stems cells and iPS cells (or other Old World monkey(s)) chromosomes based on genetic sequences selected from the recently sequenced macaque genome (Gibbs, 2007, Science 316:222-234). In such embodiments, regions homologous to human 12 and 17 are selected, for example, from monkey chromosomes 11 and 16, which have demonstrated trisomy in macaque stem cell lines. Control slides from normal monkeys are provided for hybridization studies on, for example, rhesus macaque embryonic stem cells, as has been described for human stem cells. Other regions may also be used based on critical region human homologies. In some embodiments, assays of the present invention comprise slides for screening embryonic mouse stem cells, for example, with probes for the critical regions of chromosomes 8 and 11 as well as others, such as 6, 12, X or Y, that were observed in studies and/or which may be discovered following changes in, for example, media, culture techniques, oxygen levels, etc.

In some embodiments, methods and systems of the present invention are of particular importance to commercial and/or private stem cell screening facilities, for example, cell banks. Cell banks routinely propagate stem cell lines, expand cultures, pool them, and aliquot the cells into different vials for storage (e.g., freezing) for future use. If several parallel cultures from the same embryonic stem cell line at the same passage are pooled, and the pool is found to be normal, then it is assumed that all of the frozen lots that will be expanded later are also normal. Assuming, for example, that five cells in 10,000 have trisomy 12, then by chance three of the lots would be normal while one lot might have two and another lot three cells with trisomy 12. Although the trisomic cells are only a fraction of the total, they could preferentially survive the normal attrition associated with the freezing and thawing process. If the lot of cells comprising a few trisomic cells is expanded then, for example, ten passages later there could be several cultures with 100% trisomy 12 even though the parallel cultures (e.g., cultures from different lots) are normal.

To address such a concern, testing of each lot deriving from a single frozen vial at the second passage after thawing, using the critical region test of the present invention (for example, onsite at the cell bank) requires few cells and does not compromise the expansion process. As such, it can be established whether or not the cell line is normal before wasting resources (e.g., time, money, reagents, etc.) in future expansion. This preliminary testing is not expected to eliminate the need for complete cytogenetic analysis every, for example, ten to fifteen passages (e.g., to ensure no other problems exist), but the preliminary testing serves to minimize the likelihood of expanding cell lines with aggressive chromosome changes like trisomy 12 and/or 17 or other changes described herein or later discovered.

In some embodiments, methods and systems of the present invention provide parameters for screening of cell lots by, for example, cell banks as described above. In some embodiments, one abnormal cell in 200 (0.5%) would require the rechecking of cells at the second or additional (e.g., ten) passage(s). If two or more cells in 200(>1%) are found to have a critical region abnormality, then it would be recommended that the cell lot not be used and a different parallel cell lot should be expanded and tested. If the cell lines do not contain a trisomy of the critical regions of, for example, relevant chromosomes after the subsequent passage(s), then the lines are anticipated to be normal for at least 10-15 more passages.

Testing for critical region anomalies is not limited to use by cell banks, but is also applicable for individual investigators who freeze down single cultures without pooling. As cell attrition is associated with freezing and thawing processes, a few cells with trisomy that were in such low concentration as to be non-detectable prior to freezing, could have a preferential survival advantage compared to the cytogenetically normal cells. Investigators will benefit from checking the cultures two or three passages after thawing by using the critical region test to ensure that their cell line does not have the trisomies screened for before the cultures are passaged further, thus saving time and resources by not working on abnormal stem cells.

### Detection of Critical Regions

Experiments conducted during the development of embodiments of the present invention identified a finite number of chromosomal regions that may be used to identify most chromosomal aberrations of interest. Thus, the present invention provides compositions and methods for assessing these critical regions. For example, in some embodiments, the present invention provide one or more probes useful in detecting these regions using the methods described herein or using any method known in the art.

It is generally recognized that embryonic stem cells, like all cells, are prone to develop chromosome aberrations in culture. Perhaps it had been assumed prior to the current emphasis on cytogenetic testing, that these cells, being young and protected from environmental insults, would be immune to such errors. However, based on cytogenetic analysis of approximately 700 human and 230 mouse embryonic stem cell lines from many different sources, experiments found that the range of chromosomal errors to which ES cells are prone is limited to a few sentinel chromosomes. Once cells develop trisomy or partial trisomy of one of these chromosomes, they tend to develop more extensive aberrations, similar to what occurs with cancer cell lines.

The key chromosome changes in human embryonic stem cells involve primarily chromosomes 12 and 17, followed by recurrent chromosome errors involving trisomy of chromosome 20 or extra copies of the long-arm of chromosome 1, with abnormal cells containing aberrations of chromosomes 13 and 8 (but not 12 or 17) least frequent. Although chromosome errors are common in all dividing cells, most are eliminated and do not lead to abnormal clones. However, once a mitotic accident produces a cell with an extra copy of chromosome 12 and/or 17 in an hESC line, this cell has a proliferative advantage that enables it to completely replace the normal cells in 10 to 15 passages. Human embryonic stem cell lines with an extra copy of chromosome 12 or 17 are indistinguishable from cell lines with a normal chromosome complement in terms of cell morphology, protein expression, and maintenance of pluripotency. When cells with trisomy 12 and/or 17 are injected into a nude mouse, instead of the usual benign teratomas that are expected when normal hESC are tested in this manner, one sees invasive teratocarcinomas which develop further chromosome abnormalities.

Since use of embryonic stem cells for research or therapeutic purposes is compromised by the presence of a trisomic chromosome aberration that dominates the genome, identifying these most frequent problems allows one to screen and determine whether a cell line is compromised. If such an aberration is present in only one or two cells, using an earlier passage of the same embryonic stem cell line can eliminate the problem. However, once the abnormality is present in all of the cells, most of the previously banked cultures are useless, so that a great many frozen cultures must be discarded until one finds a passage of normal cells that were banked before the aberration occurred.

Methods described herein allow for early detection of sentinel chromosome aberrations in embryonic stem cell lines, to prevent expanding compromised cultures whose maintenance is expensive and labor intensive. While there may be many rare aberrations, experiments conducted during the development of embodiments of the present invention determined that identification of a finite number of critical regions on one, two, or a few chromosomes, allows one to select and eliminate comprised cell lines. As a result of analyzing approximately 700 human and 230 mouse embryonic stem cell lines, of which 225 and 143, respectively, were abnormal, the most effective regions have been identified. For example, in human embryonic stem cell cultures these involve chromosomes 17, 12, 1 long-arm, 20, 13, and 8. In the mouse embryonic stem cell cultures, the key chromosomes are 8, 11, X, Y, 6, and 12. In some embodiments, the single most frequent aberration is identified. This can eliminate, for example, a majority of the problematic clones. In some embodiments, two of the most frequent aberrations are identified. In some embodiments, three are identified. By using an assay with reagents that detects only the limited markers (e.g., an assay that consists of reagents for these and only these markers), resources are saved, while maintaining the informative value needed to screen for desired and undesired clones.

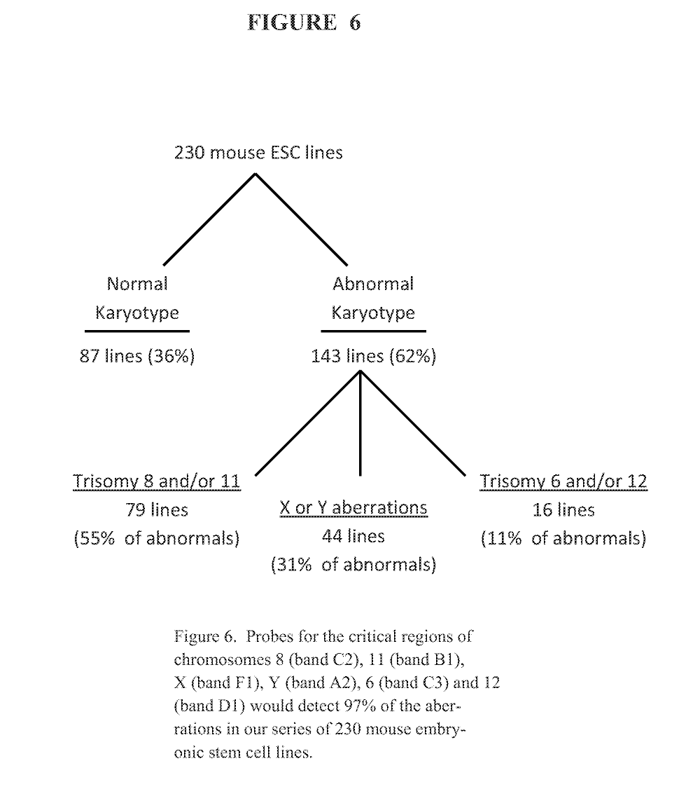

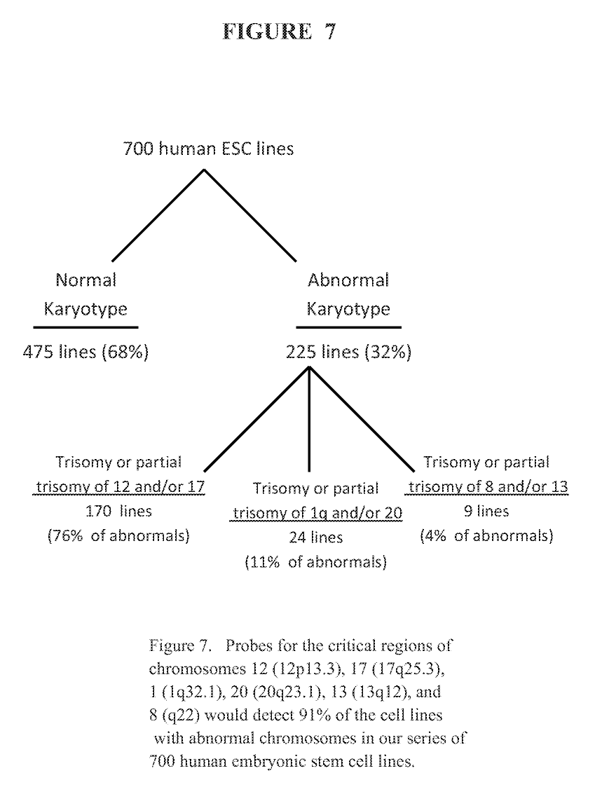

Since most of the human cells with aberrations of chromosomes other than 12, 17, 1 and 20 also had aberrations of other chromosomes, in some embodiments, there is no need to design specific probes for their detection (see FIG. 7). For example, there were 5 lines with trisomy of chromosome 7, but these all had trisomies of 12 or 17, and thus the line would have been identified as being abnormal by a 12/17 probe combination. However, there were 9 lines with full or partial trisomies of chromosomes 13 and 8 that would not have been detected with either the 12/17 or 1/20 probe combinations. The same holds true for other animal species (see e.g., FIG. 6 for murine cell lines).

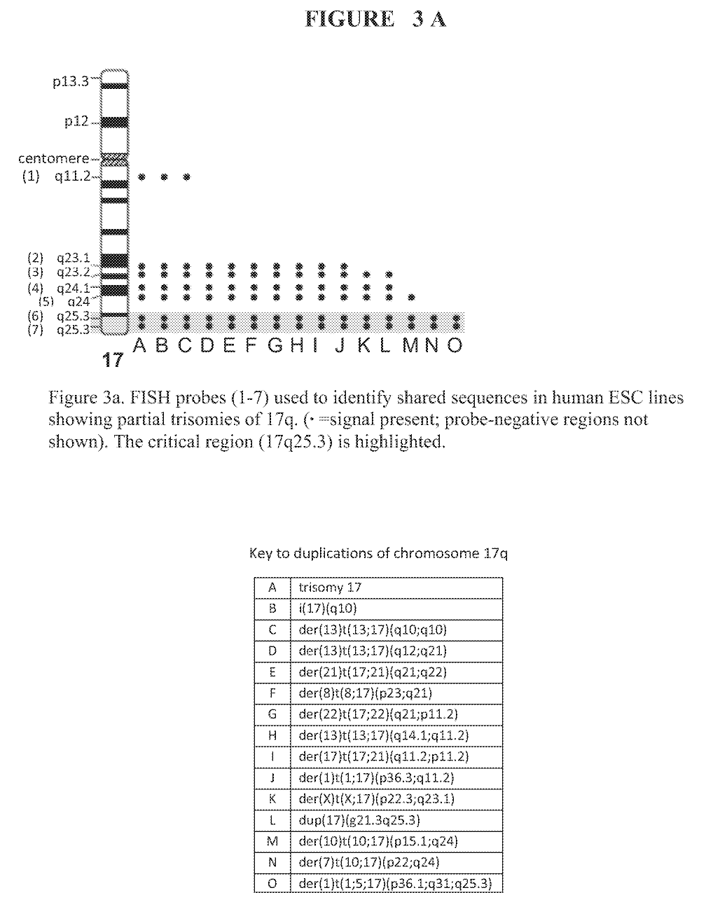

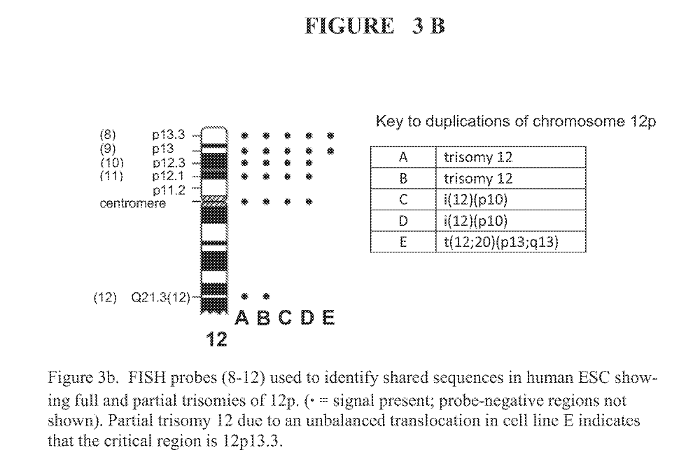

In some embodiments, ideal probes are those that are selected by determining the smallest region of a chromosome that yields the same effect as trisomy for the whole chromosome, as defined by the ability of cells with the abnormal karyotype to replace the normal cells in the cell line. Several exemplary target regions and probes are described below. The compositions, methods, and kits of embodiments of the present invention may use one or more of these target regions or probes. In some embodiments, probes are designed to span an entire critical region. In other embodiments, a probe binds to a sub-portion of a critical region, such as a gene sequence associated with the region. In other embodiments, the probe extends beyond the critical region, but includes at least a portion of the critical region. A plurality of probes may be used to span one or more regions. Critical regions can be selected narrowly as those regions that are always found to be present in duplications or translocations or may be selected more generally. For example, referring to FIG. 3a, a critical region may be identified as 17q25.3. In other embodiments, the critical region may include sequences from the q23 and/or q24 regions.

### Human Cells

Critical Region in Chromosome 17

The following table demonstrates the method of selection that was used to identify potential candidates for the critical region of chromosome 17 that leads to the proliferative advantage in hESC lines. This was based on cytogenetic analysis of 28 human embryonic stem cell lines with very small to large partial trisomies of the long arm of chromosome 17, with break points of the trisomic regions ranging from 7q11.2 to 17q25.3

| Break-points | Recipient chromosomes for partial trisomy 17 |
|---|---|
| Centromere = 2 | 1p36.3; 1q42; 5q26.1; 5q33.3; 5q31; 7p22.3; |
| Band q11.1 = 1 | 8p23; 10p15.1 13q12; 13q13; 13q14; 13q14.1; |
| Band q11.2 = 6 | 21p11.2; 17q23; 20q10; 21q11.2; 21q21; 21q22; |
| Band q12 = 1 | 22q11.2; 22q13.1 |
| Band q13.1 = 1 | |
| Band q21 = 4_{—} | |
| Band q21.3 = 3 | |
| Band q22 = 4 | |
| Band q24.1 = 1 | |
| Band q25 = 1 | |
| Band q25.1 = 2 | |
| Band q25.3 = 2 | |

As the above data show, although part of the long-arm of chromosome 17 can translocate to virtually any recipient chromosome, a probe for region 17q25.3 will identify all full and partial translocations. This was shown by hybridizing these cell lines with a battery of DNA probes spanning the long arm of chromosome 17 (see FIG. 3a) which revealed that bands 17q25.2 to 17q25.3 define the region common to all of the partially trisomic regions, and which therefore can be termed a critical region. This showed that only probes for 17q25.2 and 25.3 detected not only all trisomies, but all partial trisomies including one that was too small to be identified with G-banded chromosome analysis. It also detected partial trisomy 17q25.3 in a cell line containing what appeared to be a balanced three-way translocation, relative to which it had been difficult to explain why a balanced translocation resulted in a proliferative growth advantage resulting in replacement of all the normal cells. Compared to commercially available probes targeting the centromere and Her-2/neu gene in band 17q11.2-q12, the critical region probe (17q25.3) was more sensititve in detecting partial trisomy 17, and was able to detect partial trisomy in eight of the cell lines that would have been missed by using the commercial probes.

The fact that band 17q25 is the primary critical region in human embryonic stem cells is most unexpected, since chromosome 17q21 harbors genes such as HER-2/neu, which is amplified in breast and other cancers, and which is adjacent to a gene for the epidermal growth factor receptor (EGFR). Genes distal to 17q21, such as BRACA1 and the retinoic acid receptor (RARA) were also initially suspected to be amplified in human embryonic stem cells demonstrating a growth advantage. Thus, finding the critical region to be at 17q25 was surprising.

Critical Region in Chromosome 12

The short-arm of chromosome 12 rarely breaks except at the centromere, in which case it gives rise to an isochromosome (two copies of the short-arm and no long-arm). Isochromosomes of chromosome 12, as well as trisomy 12, are diagnostic findings in human germ cell tumors. Experiments conducted during the development of embodiments of the present invention have identified two translocations in chromosome 12 that identify sequences of interest for analysis. One involved a translocation of 12p13.3 with 20q13.1, and the second involved translocation of extra unidentifiable material on the 12 long-arm which turned out to contain the 12p13.1 sequence.

Hybridization of a battery of chromosome 12 probes specific for the short arm of chromosome 12 identified the common region 12p13.3 as a critical region on chromosome 12 (See FIG. 3b). The probe for this region proved successful in identifying not only full trisomy 12 and the more common isochromosome 12p, but a smaller partial trisomy of this region resulting from a translocation. The critical region probe (12p13.3) proved to be of superior sensitivity to commercial probes targeting the centromere of chromosome 12, and was able to detect partial trisomy resulting from isochromosome formation as well as partial trisomy resulting from translocation that the commercial probe would have missed.

Critical Regions in Chromosomes 1 and 20

The next most frequent aberrations in humans involve trisomies of chromosome 20 and the long arm of chromosome 1. The critical region in 20 is q13.3 based on its presence in translocations as well as finding several cases with isochromosomes of the 20 long-arm replacing the normal chromosome 20, thereby resulting in an extra copy of the long-arm with the critical region.

Experiments conducted during the development of the present invention have identified 15 rearrangements involving the long arm of chromosome 1. These have involved translocations with chromosomes 4, 11, 15, 18 involving break points q11, q12, and q21 resulting in extra copies of the distal 1 long-arm. However, the most significant information came from insertions or duplications of chromosome 1 involving extra copies of the regions marked by bands 1q22-q24, q21q32, q21q42, and 1q21q43, indicating that this is a critical region. Therefore, a probe spanning the regions 1q22 and 1q43, can detect cell lines with isochromosome 1 or extra copies of the chromosome 1 long-arm. This is supported by translocations involving 1q32, indicating that a critical region for detecting copy number, that will also detect all of the small 1q insertions seen, is 1q32.

Critical Regions in Chromosomes 8 and 13

Based on three cells with translocations resulting in partial trisomy 13, the critical region was determined to be 13q12, while two cells that had partial duplications of chromosome 8 indicated that the critical region for detecting all full and partial trisomies of chromosome 8 is 8q22. It should be noted that probes for two expected regions in these two chromosomes, namely 13q14, the site of the Rb suppressor gene, and 8q24, the site of the C-myc oncogene, would not identify all partial trisomies.

To summarize the ability of the above described critical regions to detect full and partial trisomies, a detection assay comprised of a slide or other system containing probes or other reagents for 12p13.3 and 17q25.3 would pick up 76% of all cells with extra copies of chromosomes 12 and 17, including full and partial trisomies (170 hESC lines). Adding a probe combination for bands 1q32 and 20p13.3 would detect an additional 11% of abnormal cell lines (24) with full or partial trisomy of the long-arm of chromosome 1 and/or full or partial trisomy of chromosome 20 that did not have trisomy 12 or 17. If a probe combination of 13q12 and 8q22 is used to identify lines with full or partial trisomies of chromosomes 8 and/or 13 that were not identified as abnormal by the other probe combinations, an additional 9 cell lines (4%) would be rejected. Using the 12/17 and 1/20 probes alone will identify 87% of abnormal cultures which should not be expanded, while probes for the 13 and 8 will only identify an additional 4%. Thus, use of a limited number of probes provides tremendous informative value.

### Mouse Cells

The critical regions in the mouse embryonic stem cell cultures were determined in the same manner. Trisomy 8 is the most frequent aberration in mouse embryonic cell lines, and is associated with a proliferative growth advantage. Experiments have detected trisomy 8 in early passages, and even in cell lines shortly after purchase from a cell bank. Injection of embryonic stem cells containing trisomy 8 into a developing mouse embryo does not interfere with normal embryogenesis, except that the resulting chimeric mice tend to develop tumors at an early age, perhaps because of the homology between mouse chromosome 8 and human chromosome 8, since trisomy 8 is a frequent occurrence in human tumors. Also, although injecting embryonic stem cells containing trisomy 8 results in chimeric mice, the trisomy 8 cells are not found in the germ line. Also, like the chromosome aberrations in human ESC, the mouse chromosome aberrations do not result in any morphological change nor do they compromise the pluripotency of the embryonic stem cells. Trisomy 11 is also one of the most common chromosome changes in mouse embryonic stem cells and is frequently found together with trisomy 8. Mouse chromosome 11 has been shown to have partial homology with human chromosome 17.

Critical Regions in Chromosomes 8 and 11

In order to determine the critical region, 33 cell lines with full and partial trisomies of chromosome 8 were analyzed. This resulted in determining that the critical region was at band C2-3. Mouse cell lines with trisomy of this region, or with partial trisomy due to a duplication or unbalanced translocation involving 8C2-3, demonstrated the adaptive growth advantage in culture associated with trisomy 8. The critical region in chromosome 11 was based on evaluating 21 cell lines with trisomies or partial trisomies due to unbalanced translocations, and was found to involve band B-1. Using a slide with probes for chromosomes 8 and 11 would detect 79 (55%) of abnormal mouse cultures.

Other Chromosomal Regions

Sex chromosome aberrations were particularly frequent in the mouse cultures, accounting for 45 of the 143 abnormal cultures that did not have trisomy 8 or 11. Entire cell lines with a single X chromosome were not unusual, and many cell lines had co-existing normal 40, XY lines with a 39, X line due to Y loss. On the basis of unbalanced translocations between an X or Y that involved different autosomes, the critical region on the X chromosome is region XF1, while the critical region on the Y chromosome is YA2. Using probes for these regions would detect an additional 31% of the abnormal cultures with extra or missing sex chromosomes, and would also identify culture contamination resulting in mouse embryonic stem cell lines with both male and female cells, of which we detected 8 lines.

The significance of the sex chromosome instability in the mouse ESC lines is unclear. The human lines rarely lost X or Y, although a few lines gained an extra copy of the X chromosome while only two lines acquired an extra Y. Also, the X and Y chromosome aberrations in the human cells usually occurred in conjunction with aberrations of 12 or 17, with only two occasions in which it was the sole aberration. Cultured cancer cells tend to lose the Y, as do leukemic bone marrow cells, whereas Y loss did not occur in the human embryonic stem cells despite its frequency in the mouse embryonic stem cell lines. Perhaps the difference in the sex chromosome distribution of the normal mouse ESC cells is relevant to the differential loss of sex chromosmes in the mESC, since the sex of the normal mouse lines was mostly male (with almost 6 times as many normal male lines as normal female lines). In contrast, the normal human embryonic stem cells had almost 1/3 more females than males, which is significantly different than the human sex ratio at birth.

In any case, if used to detect aberrations in mouse embryonic stem cells, the 8/11 probe combination followed by the using the X/Y probe combination would identify 86% of mouse ESC aberrations. An additional 11% of abnormal lines would be identified with probes for bands C3 on mouse chromosome 6, and D1 on chromosome 12, which were the only other chromosomes that were often found to be trisomic in cell lines without trisomies 8 or 11 or aberrations of the sex chromosomes.

### EXPERIMENTAL

The following examples are provided in order to demonstrate and further illustrate certain embodiments and aspects of the present invention and are not to be construed as limiting the scope thereof.

### Example 1

### FISH Probes in hESC Cultures for Chromosomes 12 and 17

This example describes the identification of exemplary probes for use in embodiments of the present invention.

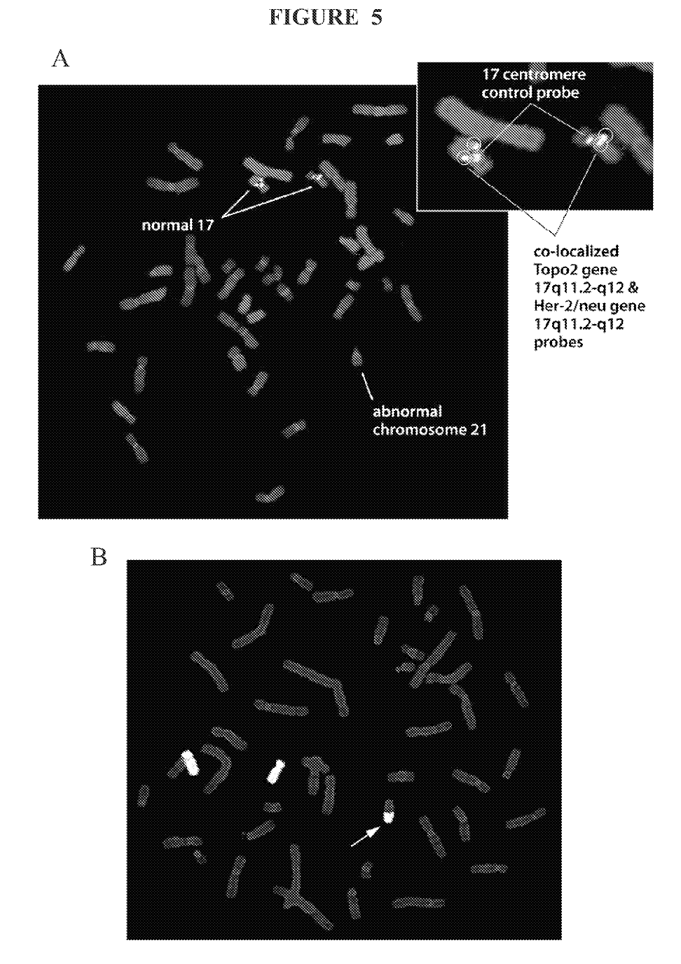

In some embodiments, FISH probes are used on interphase nuclei to detect low level clones with complete or partial trisomy 12 and/or 17 on cultures determined to be normal by routine cytogenetics. Recent successes have been realized at detecting some unusual partial trisomies of 17q and 12p in metaphase cells, using a commercial probe combination of the Her-2neu gene at 17p11.2 with the 17 centromere for chromosome 17q and TEL on chromosome 12p13. As shown in FIGS. 4-5, a partial trisomy was suggested by standard cytogenetics, but the source of the extra material was unknown and not identified by the standard probes used for detecting trisomy 17. However, use of a chromosome 17 painting probe (FIG. 5B) identified the extra material as being derived from chromosome 17. FISH probes to known hESC cell lines with different size trisomies for chromosome 17q, that would detect a common and probably smaller region on chromosome 17q, are not currently available, but the development of such probes to provide a sensitive target for detecting small partial trisomies is an embodiment of the present invention (FIGS. 3 and 5).

After defining the smallest genetic sequence that will identify whole chromosome trisomy as well as cryptic insertions leading to extra copies of the critical region, probes were constructed so that copy number of these chromosomes or their critical regions could be detected in non-dividing (interphase) nuclei. By manual or automated screening of the cells that have been hybridized with these probes, it is possible to assess the number of copies of extra chromosomes or their critical regions to a sensitivity of 0.5% (by counting 200 cells) or to 0.1% by automated evaluation of 1000 cells.

As an exemplary experiment, fixed hESC cells are resuspended in 3:1 methanol: acetic acid, dropped on slides and air dried. Slides are pretreated at 37° C. for 30 min. in 2×SSC followed by digestion in pepsin for 5 to 10 minutes at 37° C. (Vysis, 25 mg Protease I in 50 ml Protease Buffer), rinsed, and dehydrated in a graded alcohol series.

Three ul of each probe (warmed to room temperature), with or without blocking DNA, are placed on each of the prepared slides. A small coverslip is placed over each hybridization area and each area is sealed with, for example, rubber cement. The slide is co-denatured for 2 to 5 minutes at 75° C. using, for example, a pre-warmed TruTemp heat block, and then hybridized in a humidified hybridization chamber in the dark overnight at 37° C. Rubber cement and coverslips are removed, and the slide is placed in a jar of pre-warmed stringency wash (e.g., 0.2-2×SSC w/0.3 NP40) for 2 minutes at 75° C. Slides are dried, DAPI counterstain (Vysis, II cat# 320804831) is applied, and the slides are coverslipped. Interphase nuclei are scored according to guidelines specific for ESC that have been established based on extensive experience. For example, a potential problem is indicated if a single signal is seen in 200 cells.

### Example 2

### Construction and Validation of FISH Probes Designed from BAC Clones Spanning the Critical Region

As an exemplary experiment, FISH probes are constructed from BAC clones localized to the 17q critical region. An effort is made to choose BAC clones that cover and overlap the area of interest. The same is done for BAC clones for the 12p region to help narrow down the smallest common region of interest. Each probe is localized to normal chromosomes to ensure hybridization to the correct chromosome and to detect possible cross hybridization using reverse DAPI. Analytical specificity and analytical sensitivity for each probe is tested against the appropriate cell population to ensure that each probe meets acceptable standards. Probes that fail to meet acceptable standards are considered unreliable, and are eliminated from the probe panel. To determine which probe is more sensitive in detecting partial trisomy 17q, all probes are hybridized to the cell lines containing partial trisomy 17q undetectable by the Her2/Neu gene probe.

BAC clones are selected, for example, from listings at the NCBI (e.g., http://www.ncbi.nlm.nih.gov/genome/cyto/hbrc.shtml) or from known distributors of BAC clones (e.g., http://bacpac.chori.org, www.resgen.com/resources/index.php3, www.sanger.ac.uk/Teams/Team63/CloneRequest).

Isolation and purification of BAC Clone DNA is carried out using, for example, the PureLink™ HiPure plasmid DNA Purification kit and in combination with PureLink™ HiPure BAC Buffer kit (cat#, K2100-02 and cat #, K2100-18, Invitrogen, Carlsbad, Calif.) and according to the manufacturers instructions. Integration of amine-modified nucleotides into BAC DNA by nick translation according to manufacturers' instructions, using the FISH Tag™ DNA Kit (cat #F32951 Invitrogen, Carlsbad, Calif.), Vysis (DesPlaines, Ill.) or a similar nick translation kit is carried out.

The Amine-modified DNA with Fluorescent Dye is labelled according to the manufacturer's directions.

Single probes are prepared by resuspending the dye-labelled DNA in TE buffer (final concentration 4 ng/uL). To prepare double or triple probe cocktails, probes are combined with different dye-labelled DNA 1:1:1 in TE buffer (final concentration 4 ng/uL) and vortex. To prepare a Working Probe, 2.5 uL of probe is placed in 1 uL 20×SSC and 6.5 uL formamide in a microcentrifuge tube. The Working Probe is denatured at 72° C. for 5 minutes and placed on ice.

Analytical Sensitivity and Specificity for each probe is determined using, for example, NCCLS probe validation guidelines. Successful detection of partial trisomy is when at least one probe accurately detects the partial trisomy not detectable by the Her2/Neu probe.

PNA probes are constructed using sequences within the chromosome 12 and 17 critical regions. Like the others, these probes are visualized with fluorescent or non-fluorescent signals, and are placed on a substrate to which the test cells are added and hybridized (as shown in FIG. 2B). The hybridization procedures for the PNA probes are modified as described in Peptide Nucleic Acids Methods and Protocols, edited by Peter E. Nielsen (Humana Press, New Jersey) 2002.

### Example 3

### Critical Region Identification

This Example describes exemplary methods used for identification of critical region target locations on human chromosomes 12 and 17.

### Selection of Target Cells

Target Cell Selection: 22 Target cell lines (Target Cells) were selected from previously karyotyped hESC lines harboring small to large trisomies of chromosomes 12 and 17 on the long arm of chromosome 17 and short arm of chromosome 12 respectively. In addition, 8 cases with unidentifiable partial trisomies were also selected for study (See FIGS. 3a and 3b). While many of the unidentifiable partial trisomies were thought to be derived from chromosomes 12 and 17 they could not be unequivocally identified by G-banding alone. The goal was to show that these small trisomic regions were derived from chromosome 12 or 17, and could be successfully identified using synthesized DNA probes. These identified regions provide a more precise critical region that is used as target for screening partial trisomies using an interphase FISH probe or other approaches. In all cases, the cell lines used in this example refer to fixed cells pellets derived from cell lines and not live cell cultures.

Selection of BAC Clones

Thirteen DNA probes specific for the regions of interest on chromosomes 12 and 17 were constructed from BAC clones (see Table 2) by routine nick translation using digoxigenin and biotin labeled nucleotides. Five DNA probes specific for the short arm of chromosome 12 (probes 8-12, FIG. 3b) and one probe specific for the long arm of chromosome 12 were constructed. Seven probes specific for the long-arm of chromosome 17 were also constructed (probes 1-7, FIG. 3a). BAC clones were strategically selected to blanket the partially trisomic regions of chromosomes 12 and 17 in order to identify a common critical region that could be used as a target for screening partial trisomies. Following Nick translation, the labeled DNA was resuspended in routine hybridization buffer containing 70% Formamide. In some instances, it may be desired to use Human cot-1 DNA in the hybridization buffer to suppress repetitive DNA sequences.

TABLE 2
List of BAC clones used for constructing FISH probes

| | | |
|---|---|---|
| | BAC Clone | |
| | Registry | Chromosome |
| Probe Number | Number | Band Location |

Chromosome 17 BAC clones

| | | |
|---|---|---|
| 1 | RP11-398A1 | 17q11.2 |
| 2 | RP11-550K23 | 17q23.1 |
| 3 | RP11-561K8 | 17q23.2 |
| 4 | RP11-489G5 | 17q24.1 |
| 5 | RP11-52B5 | 17q24 |
| 6 | RP11-467J3 | 17q25.3 |
| 7 | RP11-117P2 | 17q25.3 |

Chromosome 12 BAC clones

| | | |
|---|---|---|
| 8 | RP11-359B12 | 12p13.3 |
| 9 (omitted) | RP11-69M1 | 12p13 |
| 10 | RP11-69C13 | 12p12.3 |
| 11 | RP11-64N21 | 12p12.1 |
| 12 | RP11-753N8 | 12q21.3 |

Probe Localization and Robustness of Signal

All probes were localized to normal human metaphase chromosomes using routine fluorescence in situ hybridization techniques (FISH) and reverse DAPI chromosome identification using Applied Imaging digital imaging software. Each of the thirteen probes was successfully localized to the expected chromosome band location with the exception of probe 9. Probe 9 unexpectedly hybridized to chromosome 10 and did not show the expected hybridization to chromosome 12. For this reason, probe 9 was eliminated from the study. All probes demonstrated robust and readable fluorescent signals though some were better than others. Both dig and biotin labeled probes worked equally well on both metaphase and interphase cells. However, the chromosome 12 probes showed some slight cross-hybridization which could be controlled by adjusting the stringency wash. In all cases both sets of probes were readable with the exception of probe 8 in some instances.

Establishing the Probe Order

Before using the probes on cell lines with abnormal chromosomes, probe pairs for 12 and 17 were hybridized to metaphase cells from karyotypically normal human cell cultures in order to localize the position of each probe relative to one another, especially when the two probes selected were in the same band. The probe order and localizations successfully matched the probe order deduced from UCSC Genome Browser (genome.ucsc.edu). Each probe was assigned a probe number reflecting their relative order (See probes 1-13 in Table 1).

### FISH Mapping to Determine the Critical Regions

Hybridization and Probe Detection

Separate studies were conducted using chromosomes 12 and 17 specific probes to determine their respective critical regions. In each case, pairs of probes labeled with different epitopes (dig or biotin) were resuspended in routine hybridization buffer, denatured using heat and formamide, and briefly pre-annealed before being applied to previously denatured slides containing the target cells. On other occasions the probe and slide were co-denatured under optimized conditions which performed equally well, thus providing a second hybridization option. Following denaturation, slides were hybridized overnight at 37 C. and subsequently washed in an optimized 2×SSC stringency wash to remove excess and loosely bound probe. A cocktail mix of secondary fluorescent antibiodies including Rhodamine anti-Digoxigenin and Alexafluor 488 Streptavidin Conjugate were used to detect the dig and biotin labeled probes respectively.

### Slide Evaluation and Scoring

Metaphase Cell Analysis

Slide preparations were examined using a fluorescence microscope equipped with the appropriate filter sets matched to the excitation and emission spectra of the antibody fluors. DAPI staining was performed on metaphase chromosomes to identify the regions of partial trisomy on chromosome 12 or 17 characteristic of each particular cell line. Using a dual band pass filter to visualize dig specific Rhodamine (red) signals and Streptavidin (FITC) specific Alexafluor 488 (green) signals, each probe was mapped to the partially trisomic region of each cell line, as well as to the eight lines with an unidentifiable trisomic region. Each map point was determined by detecting presence or absence of red or green signals on the trisomic region of interest (See FIGS. 3a and 3b). By mapping presence or absence of the specific probes, it was possible to determine whether or not a common region (critical region) was present in all of the test cell lines that could be used as a target for an interphase FISH screening probe. In addition the battery of probes were able to determine that the previously unidentifiable trisomic regions in 7 of the 8 cells lines were definitively derived from the long arm of chromosome 17, and 1 was derived from chromosome 12p, emphasizing the increased sensitivity of FISH based testing over G-banding.

Interphase Cell Analysis

20-50 interphase nuclei were examined to ensure that the interphase FISH signal patterns were consistent with partial trisomy previously detected by cytogenetic analysis with G-banding. The interphase signal patterns were consistent with the previous cytogenetics with the exception of Case E in FIG. 3a. Because of the limited amount of test sample in this line, complicated by the low frequency of abnormal cells in that line, only ten informative nuclei could be detected by interphase analysis which was not enough cells to produce a definitive result. However, an abnormal signal pattern could be quantified in all cases in which partial trisomy was detected in metaphase, as well as in the eight cases with partial trisomies of unidentifiable origin.

Results

The results show that cell lines with trisomies and partial trisomies due to unbalanced translocations or duplications can be identified by using a probe for a region common to both large and small translocations, with the region common to all of these lines termed the “critical region.” Specific FISH probes targeted to the critical region find use as interphase detection. For example, in the present series, seven of the translocations that could not be identified by metaphase G-banding were shown with these probes to encompass the critical region of chromosome 17. FISH probes targeting these regions show robust signals in interphase nuclei and can used as a simple yet efficient screening method for detecting the most common trisomies plaguing cultured embryonic stem cells, such as trisomies of 12 and 17, 1q and 20, or 8 and 13 (or mouse trisomies 8 and 11, X and Y aberrations, or mouse trisomies of 6 and 12). Based on this study, a critical region for chromosome 17 has been established to be 17q25.3 and the critical region for chromosome 12 has been established to be 12p13.3, each of which may extend to the q and p terminal end excluding the telomeric regions respectively. It should be understood, however, that other regions may be probed as well.

Identification of the critical regions and use of probes specific for these and other critical regions enables laboratories to perform routine screening for these genetic aberrations in house, using interphase cells. In addition to the fact that probing can be done in house without extensive training, a main advantage to using these probes targeting the critical regions of chromosomes prone to develop aberrations in hESC, is that it enables early detection of these trisomies since 200 or more interphase cells can be easily scored, enabling early detection of very small populations of trisomic cells so that substitution of an earlier passage in which all of the cells are still normal can enable research to proceed on cytogenetically normal lines.

### Example 4

### Assay Systems

This example describes exemplary assay systems for testing samples for chromosomal aberrations.

Two probe delivery systems were developed, one based on liquid probe delivery and the second based on a non-liquid probe delivery, each utilizing a slide-based detection system as shown in FIG. 2.

Materials:
1) Positive control cells (Fixed cells from cell line BGOIV with 100% trisomy 12 and 100% trisomy 17) prepared by routine cytogenetic methods and available through ATCC.
2) Negative Controls cells (fixed cell pellets from normal peripheral blood lymphocyte cultures).
3) Test Samples
4) 8 mm diameter circles of probe delivery surface (e.g., Mylar, glass, polyester, etc.)
5) Slide system
6) Slide hot block

Eight 8 mm circles of glass, or other probe delivery surfaces were glued in rows of 4 on an upper probe delivery platform (e.g., microscope slide). The circles fit precisely into circular wells pre-drawn onto the lower specimen delivery platform (e.g., lower microscope slide) to allow maximum contact between applied probe and applied test sample when the upper and lower platforms are sandwiched together. In a preliminary study, other delivery surfaces tested included polyester film, smooth and rough Mylar, glassine paper, and wet media acetate. All delivery surfaces were able to deliver the probe to the target cells and showed average to excellent fluorescent signal robustness. Some delivery surfaces worked better than others. Glass, Mylar and glassine paper worked best out of the group in these experiments.

Non-Liquid Probe Delivery

Preparation of the upper probe delivery platform: Probe cocktails containing dig and biotin labeled DNA specific for the critical region of chromosomes 12 and 17 were spotted on all 8 probe delivery surfaces and allowed to dry under controlled conditions.

Preparation of the lower specimen delivery platform: A prescribed amount of positive and negative controls (fixed cells) was carefully spotted on the upper and lower corners of the probe delivery surfaces respectively. Fixed cells from two different test samples were spotted in duplicate into the remaining 4 wells located in the center of the slide.

Denaturation and Hybridization: A prescribed amount of a standard hybridization buffer was applied to each of the 8 specimen wells. The upper probe delivery platform containing pre-dried probe was sandwiched on top of the lower specimen delivery platform bringing the cells in contact with the probe delivery surface. The slide was sealed with rubber cement. In each case, the delivery surface was sealed around the target area to prevent probe evaporation. Hybridization in a moist environment without gasketing the device or sealing the target area was also performed and provided a viable alternative that performed equally well. Alternatively, each delivery surface can be pre-adhered to the inside of a bubble pack by a prefabricated gasket, which enables the gasket to serve as a seal for the hybridization sandwich during co-denaturation and hybridization. However, other gasketing techniques would work equally well, including preformed rubber gaskets that are fabricated onto the perimeter of one or both delivery platforms which act as a seal once sandwiched with its companion platform. Once sealed, the devices were co-denatured in a slide heat block and allowed to hybridize overnight according to routine methods.

Stringency Wash and Detection: Following denaturation, slides were hybridized overnight at 37 C. and subsequently washed in an optimized 2×SSC stringency wash to remove excess and loosely bound probe. A cocktail mix of secondary fluorescent antibodies was used to detect the probes according to routine techniques and the slides were subsequently examined using a fluorescence microscope equipped with appropriate filter sets.

Results: A series of experiment were performed to optimize hybridization parameters and improve the signal strength of the hybridization using different probe delivery surfaces. Glass and Mylar proved to be superior surfaces, followed by wet media acetate. Glassine paper also gave a signal, but was thought to have more applications for use in roll format (described below). All delivery methods demonstrated readable fluorescent signals of differing quality, ranging from excellent to adequate. All surfaces held up well under denaturation temperatures ranging from 70-85 C. and were viable options for delivering dried probe to the hybridization target. The analysis showed that probe delivery using dry probe using the described prototype provides a viable format for providing a bench level FISH based screening assay.

Liquid Probe Delivery:

Preparation of the upper probe delivery platform and lower specimen delivery platforms: Both the upper and lower platforms were prepared as above, with the exception that probe was not applied to the upper probe delivery platform.

Co-denaturation, Hybridization, Stringency wash and Detection: Liquid probe composed of labeled DNA and standard hybridization buffer was applied directly to each well of the lower specimen delivery platform, which contained test cells and control cells. The upper platform was sandwiched on top of the lower platform, sealed, co-denatured, hybridized, and detected as described above. The denaturation and hybridization parameters were optimized to produce robust signals suitable for analysis in both metaphase and interphase cells. As an alternate procedure, the upper platform was placed over pre-denatured probe applied to the pre-denatured specimen delivery platform and hybridized as described previously.

Results: Liquid probe delivery using the prototype proved equally feasible compared with dry probe delivery. Optimization of the denaturation parameters generated usable signals that were comparable to non-liquid probe delivery in terms of sensitivity and specificity.

Non-liquid Probe Delivery using Glassine Tape (or other paper alternatives):

Materials:

1) Test samples

2) Glassine paper

3) Lower specimen delivery platform (described above)

4) Positive and negative controls (as listed above)

Preparation of the glassine probe delivery surface: Small drops of labeled nucleotide cocktails specific for the critical regions of chromosomes 12 and 17 were spotted on glassine paper and allowed to dry under controlled conditions.

Preparation of the lower specimen delivery platform. The lower specimen delivery platform was prepared as described above.

Co-denaturation, Hybridization, Stringency Wash, and Detection: A prescribed amount of standard hybridization buffer was applied to each well of the specimen delivery platform. A small square of glassine paper containing the dried spot of DNA was cut from the glassine paper sheet and applied (DNA surface down) onto each well sandwiching the hybridization buffer. The paper was sealed with rubber cement. Other options for gasketing are possible. In one scenario, the dried labeled DNA is dispensed in tape format, where each spot has a perimeter of pre-fabricated gasketing substance holding the layers of tape together. The same sticky gaskets can be “pressed” onto the specimen delivery slide to seal the hybridization sandwich together for co-denaturation and hybridization. Following co-denaturation and hybridization, the delivery surface was removed and the slides were washed and detected using routine methods described above.

Results: Signals generated by this method were less optimal than the other methods described above. However, delivery of dried probe on paper proved to be a viable option with additional optimization.

Any of the above approaches may be automated and/or conducted in a highly multiplex fashion.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. An assay for detecting chromosomal aberrations in stem cells comprising:
a) providing:
i) a substrate,
ii) a population of stem cells to be tested for aberrations of critical chromosome regions affecting from 0.5% to 100% of the population,
iii) labeled probes to regions of a chromosome,
b) applying said stem cells and said labeled probes to a substrate,
c) performing in situ hybridization such that said probes hybridize to the chromosomal DNA regions of said cells, and
d) detecting the presence or absence of hybridization of said probes to said stem cell DNA, thereby determining the presence or absence of said subpopulation in said population.

2. The assay of claim 1, wherein said detecting comprises the detecting of critical region aberrations in unknown populations of stem cells.

3. The assay of claim 1, wherein said substrate is a slide.

4. The assay of claim 1, wherein said stem cells are human embryonic stem cells.

5. The assay of claim 1, wherein said stem cells are reprogrammed or otherwise derived stem cells.

6. The assay of claim 1, wherein said critical chromosome regions are regions on human chromosome 1, 12, 13, 17, 18, 20, or X.

7. The assay of claim 1, wherein said stem cells are non-human embryonic stem cells.

8. The assay of claim 7, wherein said non-human embryonic stem cells are non-human primate embryonic stem cells.

9. The assay of claim 8, wherein said critical chromosome regions are regions found on one or more of chromosomes 6, 8, 10, 11, 16, 17 and X.

10. The assay of claim 1, wherein said stem cells are murine embryonic stem cells.

11. The assay of claim 10, wherein said critical chromosome regions are regions found on one or more of chromosomes 6, 8, 9, 11, 12, Y and X.

12. The assay of claim 1, wherein said probe is created by peptide nucleic acids synthesis.

13. The assay of claim 1, wherein said labeled probe is a digoxigenin labeled probe.

14. The assay of claim 1, wherein said aberrations comprise the critical regions of one or more of chromosome 1, chromosome 12, chromosome 13, chromosome 17, chromosome 18, chromosome 20, and chromosome X.

15. The assay of claim 1, wherein said detection is by light microscopy.

16. The assay of claim 1, wherein said detection is by fluorescent in situ hybridization wherein probes are detected with a microscope configured for fluorescence detection.

17. A method of diagnosing a stem cell population for chromosomal anomalies comprising:
a) providing a stem cell population for testing,
b) applying to said stem cell population one or more labeled probes capable of hybridizing to one or more critical regions on one or more chromosomes in a stem cell,
c) diagnosing a stem cell population as containing chromosomal anomalies based on said hybridization, wherein said hybridization of said one or more probes detects the presence of abnormal cells present in 0.5% or less of cells in said stem cell population.

18. The method of claim 17, further comprising the step of transplanting said stem cells into an organism if said stem cell population does have a detectable chromosomal anomaly.

19. A composition comprising one or more reagents that specifically detect a chromosomal trisomy associated with human chromosomes 17q25.3 and 12p13.3.

20. The composition of claim 19, wherein said reagents consist essentially of or consist of in situ hybridization probes that specifically detect chromosomal trisomy associated with human chromosomes 17q25.3 and 12p13.3.
